(19) United States
(12) Patent Application Publication
(10) Pub. No.: US 2025/0138115 A1
(43) Pub. Date: May 1, 2025

(54) ADAPTIVE COMPRESSION SYSTEM FOR RADIO FREQUENCY COIL ASSEMBLY

(71) Applicant: GE Precision Healthcare LLC, Waukesha, WI (US)

(72) Inventors: Edward Joseph Baus, Akron, OH (US); Jana Michelle Vincent, Aurora, OH (US); Gustavo Andres Chacon-Rojas, Cleveland, OH (US)

(21) Appl. No.: 18/494,211

(22) Filed: Oct. 25, 2023

(51) Int. Cl. G01R 33/34; A61B 5/00; A61B 5/055

(52) U.S. Cl. CPC G01R 33/34084; A61B 5/055; A61B 5/4312

(57) ABSTRACT

An RF receiving coil assembly for an MRI system includes a flexible enclosure and an RF coil disposed within the flexible enclosure. The flexible enclosure is configured to be disposed on a first side of a portion of a subject to be imaged. The RF receiving coil assembly further includes an adaptive compression system. The adaptive compression system includes a flexible retaining element separate from the flexible enclosure that is configured to be disposed on a second side of the portion of the subject. The adaptive compression system also at least one band coupled to both the flexible enclosure and the flexible retaining element. The adaptive compression system includes a tension adjustment mechanism coupled to the at least one band and configured to adjust a tension of the at least one band to adjust how tight the flexible enclosure is disposed about the portion of the subject to be imaged.

### BACKGROUND

The subject matter disclosed herein relates to medical imaging and, more particularly, to an adaptive compression system for a radio frequency (RF) coil assembly for a magnetic resonance imaging (MRI) system

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During magnetic resonance imaging, when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

Current techniques or mechanisms for retaining a coil (e.g., body coil) to a patient are through the use of hook and loop fastener straps that wrap around the patient, varying rigid positioners, and/or foam supports. These straps are difficult to use, hard to position, and often degrade over time. Additionally, these straps do not effectively account for contoured surfaces such as shoulders breasts, ankles, hips, and related areas. Due to varying anatomical sizes (e.g., varying bust sizes or patient sizes) or medical conditions (e.g., having a mastectomy), straps or other positioners do not provide the best retention of the coil to the body.

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a radio frequency (RF) receiving coil assembly for a magnetic resonance imaging (MRI) system is provided. The radio frequency receiving coil assembly includes a flexible enclosure. The radio frequency receiving coil assembly also includes a radio frequency coil disposed within the flexible enclosure, wherein the radio frequency coil includes a plurality of flexible loops having a malleable conductor. The flexible enclosure is configured to be disposed on a first side of a portion of a subject to be imaged. The radio frequency receiving coil assembly also further includes an adaptive compression system. The adaptive compression system includes a flexible retaining element separate from the flexible enclosure that is configured to be disposed on a second side of the portion of the subject to be imaged. The adaptive compression system also includes at least one band coupled to both the flexible enclosure and the flexible retaining element. The adaptive compression system further includes a tension adjustment mechanism coupled to the at least one band and configured to adjust a tension of the at least one band to adjust how tight the flexible enclosure is disposed about the portion of the subject to be imaged.

In another embodiment, a radio frequency (RF) receiving coil assembly for a magnetic resonance imaging (MRI) system is provided. The radio frequency receiving coil assembly includes a flexible enclosure. The radio frequency receiving coil assembly also includes a radio frequency coil disposed within the flexible enclosure, wherein the radio frequency coil includes a plurality of flexible loops having a malleable conductor. The flexible enclosure is configured to be disposed on a first side of a portion of a subject to be imaged. The radio frequency receiving coil assembly also further includes an internal adaptive compression system. The internal adaptive compression system includes a flexible retaining element separate from the flexible enclosure that is configured to be disposed on a second side of the portion of the subject to be imaged. The internal adaptive compression system also includes cordage coupled to both the flexible enclosure and the flexible retaining element, wherein at least a portion of the cordage is disposed within the flexible enclosure. The internal adaptive compression system further includes a tension adjustment mechanism coupled to the cordage and configured to adjust a tension of the cordage to adjust how tight the flexible enclosure is disposed about the portion of the subject to be imaged.

In a further embodiment, a radio frequency (RF) receiving coil assembly for a magnetic resonance imaging (MRI) system is provided. The radio frequency receiving coil assembly includes a flexible enclosure. The radio frequency receiving coil assembly also includes a radio frequency coil disposed within the flexible enclosure, wherein the radio frequency coil includes a plurality of flexible loops having a malleable conductor. The flexible enclosure is configured to be disposed on a chest having breasts of a subject to be imaged. The radio frequency receiving coil assembly also includes an integral adaptive compression system. The integral adaptive compression system is configured to enable positioning of the flexible enclosure about breasts of different sizes. The integral adaptive compression system includes a flexible retaining element separate from the flexible enclosure that is configured to be disposed on a back of the subject to be imaged. The integral adaptive compression system also includes cordage coupled to both the flexible enclosure and the flexible retaining element, wherein the cordage includes a first net-like region of cordage within the flexible enclosure and a second net-like region of cordage within the flexible enclosure that are configured to be respectively disposed about a respective breast of the breasts. The integral adaptive compression system further includes a tension adjustment mechanism coupled to the cordage and configured to adjust a tension of the cordage to adjust how tight the flexible enclosure is disposed about the chest of the subject to be imaged.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 illustrates an embodiment of a magnetic resonance imaging (MRI) system suitable for use with the disclosed technique;

FIG. 2 is a schematic diagram of a radio frequency (RF) coil assembly to be utilized with an adaptive compression system or integrated positioning system, in accordance with aspects of the present disclosure;

FIG. 3 is a schematic diagram of a cross-section of a radio frequency coil, in accordance with aspects of the present disclosure;

FIG. 4 is a schematic diagram of a body coil having an adaptive compression system or integrated positioning system, in accordance with aspects of the present disclosure;

FIG. 5 is schematic diagram of the body coil having the adaptive compression system or integrated positioning system in FIG. 4, in accordance with aspects of the present disclosure;

FIG. 6 is a schematic diagram of a portion of the adaptive compression system or integrated positioning system in FIG. 5, in accordance with aspects of the present disclosure;

FIG. 7 is a schematic diagram of a portion of the adaptive compression system or integrated positioning system in FIG. 5 disposed about a subject to be imaged, in accordance with aspects of the present disclosure;

FIG. 8 is a schematic diagram of a portion of the adaptive compression system or integrated positioning system in FIG. 5 (e.g., with a multi-piece flexible retaining element), in accordance with aspects of the present disclosure;

FIG. 9 is a schematic diagram of a portion of a tension adjustment mechanism, in accordance with aspects of the present disclosure;

FIG. 10 is a schematic diagram of a top view of the body coil having the adaptive compression system or integrated positioning system in FIG. 4 (e.g., configured for use with breasts) disposed on a chest region (e.g., having breasts) of a subject, in accordance with aspects of the present disclosure;

FIG. 11 is a schematic diagram of a rear view of the body coil having the adaptive compression system or integrated positioning system in FIG. 10, in accordance with aspects of the present disclosure; and

FIG. 12 is a schematic diagram of a radio frequency (RF) coil assembly to be utilized with an adaptive compression system or integrated positioning system (e.g., with a flexible enclosure having openings), in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

The present disclosure provides embodiments of an adaptive compression system (e.g. integrated adaptive compression system) or integrated positioning system for retaining a body coil (e.g., a radio frequency coil assembly or radio frequency receiving coil) on a subject to be imaged. In particular, the adaptive compression system utilizes a cinching technique that utilizes elastic cordage or bands (a portion of which is integrated or disposed within a flexible enclosure of the body coil) and a flexible retaining element. When the cordage or bands are pulled, the body coil (in a flexible enclosure) and the retaining element are pulled closer together retaining the body coil to the body. The adaptive compression system is adjustable so that the body coil can be pulled as tight or as loosely as needed while the cordage layout allows for customized contours around a wide range of anatomical regions. Although discussed in the context of use with a body coil, the adaptive compression system can be utilized with other items or devices (e.g., conformal flight vests or other form-fitting clothing).

The disclosed embodiments benefit the subject to be imaged (e.g., patient) by providing a more comfortable positioning of the body coil on the body. The cordage can be loosened or repositioned as needed for imaging and patient comfort. The disclosed adaptive compression system can be oriented in different locations. The disclosed adaptive compression system can be utilized with a body coil in a single base or with modular insertable that are tailored to the patient. The adjustable size of the adaptive compression system will allow for adjustment to the tissue while allowing for free hanging of the tissue.

Compared to existing straps and positioning systems, the disclosed embodiments provide a much faster and streamlined configuration. Thus, the setup time is reduced and the throughput for use of the magnetic resonance scanner can be increased. Also, the need for use of bulker positioners is eliminated. For the healthcare providers (e.g., hospital or imaging center), as components (e.g., cordage) of the adaptive compression system degrade, they can be easily removed and replaced with new cordage. In addition, from a safety perspective, the cinched body coil can be put tighter against the body away from the bore of the magnetic resonance scanner. Thus, the body coil is less likely to heat as much (e.g., due to field inhomogeneities at the edge of the bore). This is especially useful for parts of the anatomy such as shoulders and hips that are already in close proximity to the bore.

The disclosed embodiments increase image quality due to a higher signal-to-noise ratio since the loops of the body coil will be closer to the body. The disclosed adaptive compression system is easier to maintain and to clean.

The disclosed embodiments can be utilized for a number of different applications and a number of different body designs (e.g., head, breast, etc.). For example, the disclosed embodiments can be utilized for pediatric patients due to ease of conforming around the body. In particular, the disclosed embodiments can be utilized to swaddle a neonate or child, reducing the bulk of straps and restraints that could be emotionally taxing for parents to see. In another example, the disclosed embodiments can be utilized with breasts. In particular, the body coil can be pulled closer to the body for different size patients and cup sizes. The adaptive compression system can be implemented to hold larger bust sizes more central when positioned supine or to hold a flexible body coil in place when used in the prone position. The adaptive compression system can be utilized for handling a subject who has had a mastectomy. In a further example, the disclosed embodiments can be utilized for hip or shoulder retention. In particular, the coil is pulled closer around a curved surface. The disclosed embodiments can be utilized on different body types and shapes.

In certain embodiments, a radio frequency (RF) receiving coil assembly for a magnetic resonance imaging (MRI) system includes a flexible enclosure. The radio frequency receiving coil assembly also includes a radio frequency coil disposed within the flexible enclosure, wherein the radio frequency coil includes a plurality of flexible loops having a malleable conductor. The flexible enclosure (and the radio frequency coil) is configured to be disposed on a first side of a portion of a subject to be imaged. The radio frequency receiving coil assembly also further includes an adaptive compression system. The adaptive compression system includes a flexible retaining element separate from the flexible enclosure that is configured to be disposed on a second side of the portion of the subject to be imaged. The adaptive compression system also includes at least one band coupled to both the flexible enclosure and the flexible retaining element. The adaptive compression system further includes a tension adjustment mechanism coupled to the at least one band and configured to adjust a tension of the at least one band to adjust how tight the flexible enclosure (and the radio frequency coil) is disposed about the portion of the subject to be imaged.

In certain embodiments, at least a portion of the at least one band is disposed within the flexible enclosure. In certain embodiments, the tension adjustment mechanism is coupled to the at least one band outside the flexible enclosure. In certain embodiments, the tension adjustment mechanism is configured, via the at least band, to pull the flexible enclosure and the flexible retaining element closer together about the portion of the subject to be imaged. In certain embodiments, the at least one band includes a first band and a second band coupled to both the flexible enclosure and the flexible retaining element. In certain embodiments, the flexible enclosure includes a first end and a second end opposite the first end, the first band and the second band are respectively disposed closer to the first end and the second end, and the tension adjustment mechanism is coupled to both the first band and the second band and is configured to adjust the tension of both the first band and the second band. In certain embodiments, the at least one band is configured to be removed and to be replaced. In certain embodiments, the at least one band includes twine, an elastic cord, or an elastic band. In certain embodiments, the tension adjustment mechanism includes a dial coupled to a spool, wherein the dial is configured to be turned to wind the at least one band on to the spool to tighten the flexible enclosure about the portion of the subject to be imaged. In certain embodiments, the tension adjustment mechanism includes a cord lock stopper. In certain embodiments, the adaptive compression system is configured to enable positioning of the flexible enclosure about curved surfaces of different parts of an anatomy of the subject to be imaged.

In certain embodiments, a radio frequency (RF) receiving coil assembly for a magnetic resonance imaging (MRI) system includes a flexible enclosure. The radio frequency receiving coil assembly also includes a radio frequency coil disposed within the flexible enclosure, wherein the radio frequency coil includes a plurality of flexible loops having a malleable conductor. The flexible enclosure (and the radio frequency coil) is configured to be disposed on a first side of a portion of a subject to be imaged. The radio frequency receiving coil assembly also further includes an internal adaptive compression system. The internal adaptive compression system includes a flexible retaining element separate from the flexible enclosure that is configured to be disposed on a second side of the portion of the subject to be imaged. The internal adaptive compression system also includes cordage coupled to both the flexible enclosure and the flexible retaining element, wherein at least a portion of the cordage is disposed within the flexible enclosure. The internal adaptive compression system further includes a tension adjustment mechanism coupled to the cordage and configured to adjust a tension of the cordage to adjust how tight the flexible enclosure (and the radio frequency coil) is disposed about the portion of the subject to be imaged.

In certain embodiments, a radio frequency (RF) receiving coil assembly for a magnetic resonance imaging (MRI) system is provided. The radio frequency receiving coil assembly includes a flexible enclosure. The radio frequency receiving coil assembly also includes a radio frequency coil disposed within the flexible enclosure, wherein the radio frequency coil includes a plurality of flexible loops having a malleable conductor. The flexible enclosure (and the radio frequency coil) is configured to be disposed on a chest having breasts of a subject to be imaged. The radio frequency receiving coil assembly also includes an integral adaptive compression system. The integral adaptive compression system is configured to enable positioning of the flexible enclosure about breasts of different sizes. The integral adaptive compression system includes a flexible retaining element separate from the flexible enclosure that is configured to be disposed on a back of the subject to be imaged. The integral adaptive compression system also includes cordage coupled to both the flexible enclosure and the flexible retaining element, wherein the cordage includes a first net-like region of cordage within the flexible enclosure and a second net-like region of cordage within the flexible enclosure that are configured to be respectively disposed about a respective breast of the breasts. The integral adaptive compression system further includes a tension adjustment mechanism coupled to the cordage and configured to adjust a tension of the cordage to adjust how tight the flexible enclosure (and the radio frequency coil) is disposed about the chest of the subject to be imaged.

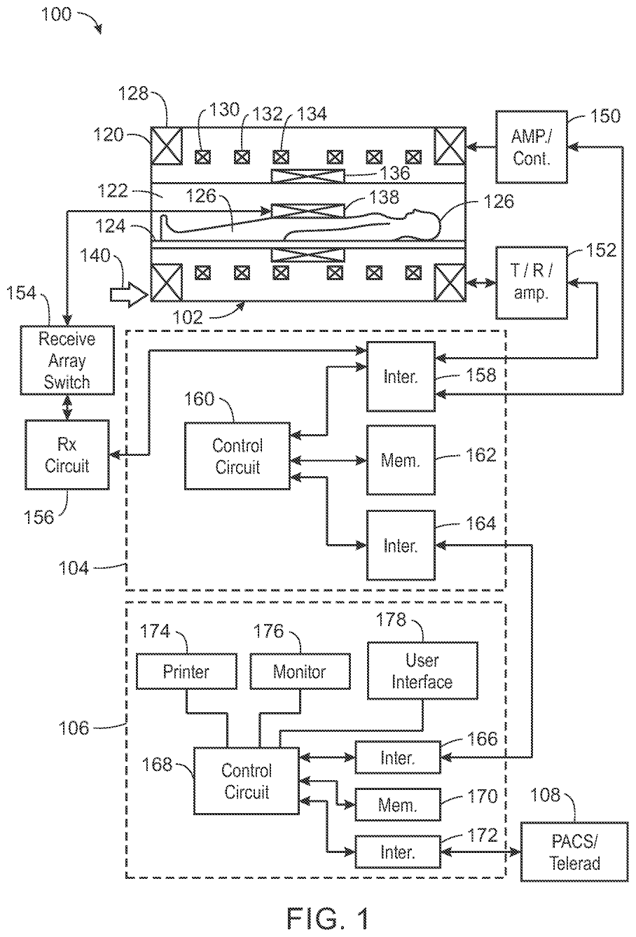

With the preceding in mind, FIG. 1 a magnetic resonance imaging (MRI) system 100 is illustrated schematically as including a scanner 102, scanner control circuitry 104, and system control circuitry 106. According to the embodiments described herein, the magnetic resonance imaging system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on- or off-site. In this way, MR data may be acquired, followed by on- or off-site processing and evaluation. While the magnetic resonance imaging system 100 may include any suitable scanner or detector, in the illustrated embodiment, the system 100 includes a full body scanner 102 having a housing 120 through which a bore 122 is formed. A table 124 is moveable into the bore 122 to permit a patient 126 to be positioned therein for imaging selected anatomy within the patient.

Scanner 102 includes a series of associated coils for producing controlled magnetic fields for exciting the gyromagnetic material within the anatomy of the subject being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., radio frequency transmit coil) is configured to generate radio frequency pulses for exciting the certain gyromagnetic nuclei within the patient. In addition to the coils that may be local to the scanner 102, the system 100 also includes a set of receiving coils or radio frequency receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak radio frequency signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, B₀. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the radio frequency coil 136. Circuit 152 includes a switching device for alternating between the active and inactive modes of operation, wherein the radio frequency coil 136 transmits and does not transmit signals, respectively. Circuit 152 also includes amplification circuitry configured to generate the radio frequency pulses. Similarly, the receiving coils 138 are connected to switch 154, which is capable of switching the receiving coils 138 between receiving and non-receiving modes. Thus, the receiving coils 138 resonate with the radio frequency signals produced by relaxing gyromagnetic nuclei from within the patient 126 while in the receiving mode, and they do not resonate with radio frequency energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the radio frequency coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the magnetic resonance imaging system 100 and to store the processed image data for later reconstruction, display and transmission. The programming code may execute one or more algorithms that, when executed by a processor, are configured to perform reconstruction of acquired data as described below. In certain embodiments, the memory circuit 170 may store one or more neural networks for reconstruction of acquired data as described below. In certain embodiments, image reconstruction may occur on a separate computing device having processing circuitry and memory circuitry.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

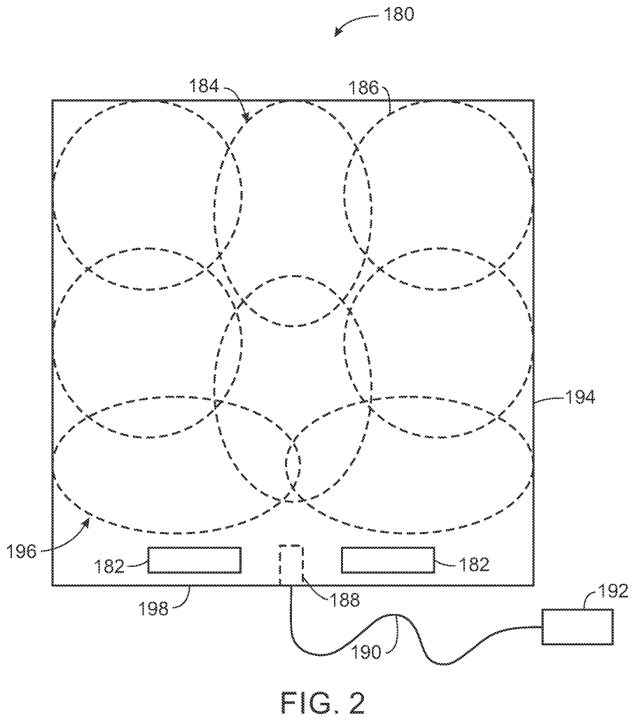

FIG. 2 is a schematic diagram of a radio frequency coil assembly 180 (e.g., radio frequency receiving coil assembly) to be utilized with an adaptive compression system or integrated positioning system. The radio frequency coil assembly 180 may be utilized in an magnetic resonance imaging system (e.g., magnetic resonance imaging system 100 in FIG. 1). The radio frequency coil assembly 180 includes an radio frequency coil 184 having a plurality of loops 186 (e.g., elements or channels). Each loop 186 is coupled to an electronics unit coupled to a coil-interfacing cable. The coil-interfacing cables of each of the loops 186 is coupled to an electrical connector interface or interface circuitry 188 (e.g., a balun such as integrated balun cable harness which may act as an radio frequency trap). The electrical connector interface 188 is coupled (via a cable 190) to a P connector 192 (e.g., port connector) that enables the radio frequency coil assembly 180 to be coupled (e.g., via wired connection) to the interface of the magnetic resonance imaging system that couples imaging components to processing components. In certain embodiments, the radio frequency coil assembly 180 may lack a wired connection and may be configured to be utilized wirelessly (e.g., for coupling imaging components to wireless components) with the magnetic resonance imaging system during an magnetic resonance imaging scan.

Each loop 186 may consist of linked resonator elements coupled to a printed circuit board module (e.g., the electronics unit). Each electronics unit may include various components (e.g., a decoupling circuit, an impedance inverter circuit, and a pre-amplifier). The radio frequency coil 184 may be designed utilizing AIR™ coil technology from General Electric Healthcare. This enables the radio frequency coil 184 to be lightweight and flexible. Each loop 186 includes a malleable (e.g., flexible) conductor that enables complex and irregular surface contours. In certain embodiments, each loop 186 may stretch (e.g., due to a liquid metal conductor). Alternatively, each loop 186 may include litz wire, a regular stranded wire, or a spiral wire woven on an extendible non-conductive support or a meandering trace. In addition, the loops 186 of the radio frequency coil 184 are transparent, thus, aiding signal-to-noise ratios.

The radio frequency coil 184 is disposed within a flexible enclosure 194 (e.g., blanket). As depicted, the flexible enclosure 194 has a rectangular shape. In certain embodiments, the flexible enclosure 194 may have a square shape or other shape. In certain embodiments, the flexible enclosure 194 includes holes or openings to increase a flexibility of the radio frequency coil assembly 180 (and the flexible enclosure 194). Each hole or opening may be radially located within a loop 186. In certain embodiments, the flexible enclosure 194 may include deformable material within. The deformable material may include foam, memory foam, expanded foam, polyurethane foam, gels such as hydrogel, cells of water, or other suitable deformable material. When the subject lies on the radio frequency coil assembly 180, the subject will sink into the deformable material and the radio frequency coil 84 may conform to the subject's unique shape and, thus, be right up against the patient's body. As depicted, the interface circuitry 188 is disposed within the flexible enclosure 194. In certain embodiments, the interface circuitry 188 may be disposed outside the flexible enclosure 194.

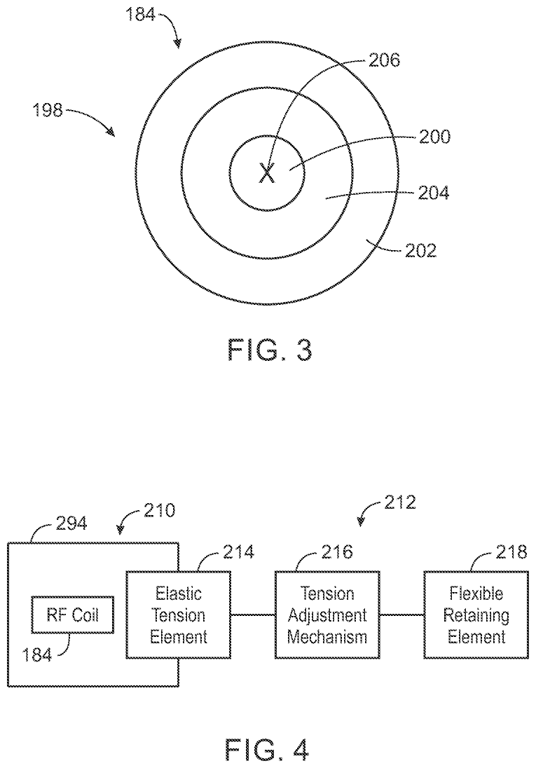

Each loop 184 includes a distributed capacitance construction. In particular, each loop 184 includes a coaxial conductor having a cross-section configured to generate exact capacitance for loop tuning at a specific frequency. For example, as depicted in FIG. 3, a coaxial coil loop portion 198 (of a loop 184) includes a round center conductor wire 200, an outer concentric shield 202, and a dielectric material 204 in between. The center conductor wire 200 may be of copper (e.g., silver plated copper) and the dielectric material 204 may be rubber, plastic, or some other dielectric material (e.g., fluoroethylenepropylene (FEP) or polytetrafluoroethylene (pTFE)). The outer concentric shield 202 may encase or otherwise surround the dielectric material 204 and center conductor wire 200 and may be comprised of braided copper or other suitable conductive material. The center conductor 200, dielectric material 204, and outer shield 202 all share a common central axis 206. Further, while not shown in FIG. 3, in some examples, an outer jacket (e.g., made of dielectric material) may surround the outer shield 202. While two coaxial conductors (center conductor wire 200 and outer shield 202) are shown in FIG. 3, an radio frequency coil loop portion may include three or more coaxial conductors, encapsulated and separated from each other by dielectric material. In certain embodiments, the center conductor wire 200 may be made of a liquid metal conductor to enable the loop 184 to be stretched.

FIG. 4 is a schematic diagram of a body coil 210 (e.g., radio frequency coil assembly 180 in FIG. 2) having an adaptive compression system (e.g., integrated compression system) or integrated positioning system 212. As depicted, the body coil 210 includes the RF coil 184 disposed within the flexible enclosure 194. The RF coil 184 includes a plurality of flexible loops having a malleable conductor. The RF coil 184 may be designed utilizing AIR™ coil technology from General Electric Healthcare. The body coil 210 may be specifically configured for use with different parts of the anatomy (e.g., head, breast, extremity, etc.). The adaptive compression system 212 is configured to enable positioning of the body coil (and the flexible enclosure 194) about curved surface of different parts of an anatomy of the subject to be imaged.

The adaptive compression system 212 includes an elastic tension element 214, a tension adjustment mechanism 216, and a flexible retaining element 218. A portion of the elastic tension element 214 is disposed within the flexible enclosure 194. Another portion of the elastic tension element 214 is disposed outside the flexible enclosure 194. The elastic tension element 214 may include one or more bands or cordage. The elastic tension element 214 may include one or more elastic cords, one or more elastic bands, or twine. The number of elastic cords or bands and the arrangement of the elastic or bands may vary depending on the application (e.g., head application, breast application, extremity application, etc.). For example, a single cord or band may be utilized when the body coil 210 is configured for used with a head. Multiple cords or bands may be utilized when the body coil 210 is configured for used with the chest or back. The elastic tension element 214 is made of magnetic resonance imaging compatible material. The elastic tension element 214 is configured to be removed and to be replaced.

The flexible retaining element 218 is separate from the flexible enclosure 194. While the body coil 210 (and the flexible enclosure 194) are configured to be disposed on a first side of a portion of a subject to be imaged, the flexible retaining element 218 is configured to be disposed on a second side (e.g., opposite the first side) of the portion of the subject to be imaged. For example, in imaging the chest region, the body coil 210 (and the flexible enclosure 294) may be disposed on the chest region and the flexible retaining element 218 may be disposed on the back region. In his scenario, the placement of the body 210 and the flexible retaining element 218 may be reversed. In another example, the body coil 210 (and the flexible enclosure 194) may be disposed on a front of a head and the flexible retaining element 218 may be disposed on a back of the head. In certain embodiments, the flexible retaining element 218 may be a single piece. In certain embodiments, the flexible retaining element 218 may be a multiple pieces. In certain embodiments, the flexible retaining element 218 may be a flat flexible piece. The shape of the flexible retaining element 218 may vary. The flexible retaining element 218 is made of magnetic resonance imaging compatible material. In certain embodiments, the flexible retaining element 218 may be made of polymer compounds (e.g., polyurethane, polycarbonate, etc.). The flexible retaining element 218 is configured to be removed and to be replaced.

The tension adjustment mechanism 216 is coupled to the elastic tension element 214 outside the flexible enclosure 194. The tension adjustment mechanism 216 is configured to adjust a tension of the elastic tension element 214 to adjust how tight the body coil 210 (and the flexible enclosure 194) is disposed about the portion of the subject to be imaged. The tension adjustment mechanism 216 is configured, via the flexible retaining element 218, to pull the body coil 210 (and the flexible enclosure 194) and the flexible retaining element 218 closer together about the portion of the subject to be imaged. The tension adjustment mechanism 216 is made of magnetic resonance imaging compatible material. In certain embodiments, multiple tension adjustment mechanisms 216 may be coupled to multiple band or cords of the flexible retaining element 218. In certain embodiments, multiple tension adjustment mechanism 216 may be coupled to a single band or cord of the flexible retaining element 218. The tension adjustment mechanism 216 is configured to be removed and to be replaced.

A variety of different mechanisms may be utilized for the tension adjustment mechanism 216. In certain embodiments, the tension adjustment mechanism 216 includes a dial coupled to a spool. The dial is configured to be turned to wind the elastic tension element 214 on to the spool to tighten the body coil 210 (and the flexible enclosure 194) about the portion of the subject to be imaged. In certain embodiments, the tension adjustment mechanism 216 includes a cord lock stopper. For example, the cord lock stopper may include a round plastic toggle stopper, a plastic cord stopper, a plastic twin hole card lock spring loaded toggle stopper, a metal cord lock spring loaded stop toggle fastener, a metal spring-loaded card stopper, or a metal spring loaded cord lock stop toggle fastener.

In certain embodiments, the flexible enclosure 194 has a first end and a second end. In certain embodiments, the elastic tension element 214 may include a first band or cord disposed more adjacent to first end of the flexible enclosure 194 (within the flexible enclosure 194) and a second band or second cord disposed more adjacent the second end of the flexible enclosure 194 (within the flexible enclosure 194). Either the first band/cord or the second band/cord may be adjusted, via the tension adjustment mechanism 216, to restrain the body coil 210 closer to the portion of the subject to be imaged by tightening it around the body. This enables the body coil 210 to be adjusted about a different body types and shapes.

In certain embodiments, the adaptive compression system 212 is configured for use with pediatric patients due to ease of conforming around the body. In particular, the adaptive compression system 212 can be utilized to swaddle a neonate or child, reducing the bulk of straps and restraints that could be emotionally taxing for parents to see.

In certain embodiments, the adaptive compression system 212 can be utilized with breasts. In certain embodiments, the elastic tension element 214 includes a first net-like cordage region within the flexible enclosure 194 and a second net-like region of cordage within the flexible enclosure 194. Each net-like region of cordage is configured to be disposed (and a portion of the body coil 210) about a respective breast of the breasts. The adaptive compression system 212 enables the body coil 210 to be pulled closer to the body for different size patients and cup sizes. The adaptive compression system 212 can be implemented to hold larger bust sizes more central when positioned supine or to hold a flexible body coil 210 in place when used in the prone position. The adaptive compression system 212 can be utilized for handling a subject who has had a mastectomy.

The adaptive compression system 212 can be utilized for other parts of the body. For example, the adaptive compression system 212 can be utilized for hip or shoulder retention. In particular, the body coil 210 is pulled closer around a curved surface.

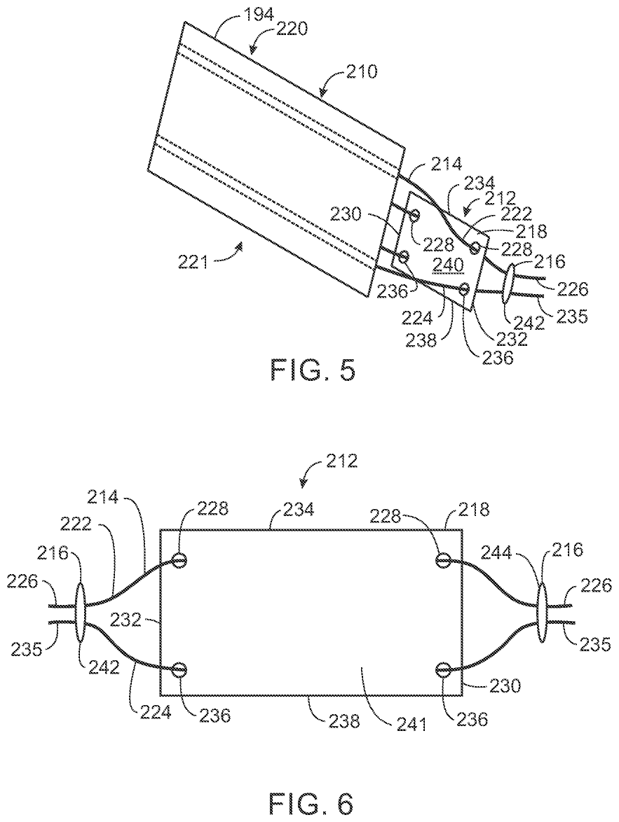

FIG. 5 is schematic diagram of the body coil 210 having the adaptive compression system or integrated positioning system 212 in FIG. 4. FIG. 6 is a schematic diagram of a portion of the adaptive compression system or integrated positioning system 212 in FIG. 5. The body coil 210 (and the flexible enclosure 194) includes a first end 220 and a second end 221 (e.g., opposite the first end 220). As depicted, the elastic tension element 214 includes a first cord or band 222 and a second cord or band 224. As depicted, the body coil 210 includes a portion of the elastic tension element 214 (of both the first cord or band 222 and the second or band 224) disposed within the flexible enclosure 194. In particular, the elastic tension element 214 includes the first cord or band 222 disposed more adjacent to first end 220 of the flexible enclosure 194 (within the flexible enclosure 194) and the second band or second cord disposed more adjacent the second end 222 of the flexible enclosure 194 (within the flexible enclosure 194).

The elastic tension element 214 is coupled to the flexible retaining element 218. In particular, respective ends 226 of the first cord or band 222 extend through respective openings 228 on opposite sides 230, 232 of the flexible retaining element 218 adjacent side 234. Respective ends 235 of the second cord or band 224 extend through respective openings 236 on the opposite sides 230, 232 of the flexible retaining element 218 adjacent side 238. As depicted, the flexible retaining element 218 is a single flat flexible piece that is configured to bend around a curved surface of a portion of a body. The flexible retaining element 218 has a first surface 240 facing the body coil 210 and second surface 241 opposite the first surface 240. A first tension adjustment mechanism 242 is coupled to both an end 226 of the first cord or band 222 and an end 235 of the second cord or band 224 beyond the second surface 241. A second tension adjustment mechanism 244 is coupled to the opposite end 226 of the first cord or band 222 and an opposite end 235 of the second cord or band 224 beyond the second surface 241. As depicted, both the first tension adjustment mechanism 242 and the second tension adjustment mechanism 244 are cord lock stoppers. For example, the cord lock stoppers may be round plastic toggle stoppers, plastic cord stoppers, plastic twin hole card lock spring loaded toggle stoppers, metal cord lock spring loaded stop toggle fasteners, metal spring-loaded card stoppers, or metal spring loaded cord lock stop toggle fasteners. In certain embodiments, a different mechanism may be utilized for the first tension adjustment mechanism 244 and the second tension adjustment mechanism 246.

The first cord or band 222 and/or the second cord or band 224 may be adjusted, via the respective tension adjustment mechanisms 242, 244, to restrain the body coil 210 closer to the portion of the subject to be imaged by tightening it around the body. This enables the body coil 210 to be adjusted about a different body types and shapes.

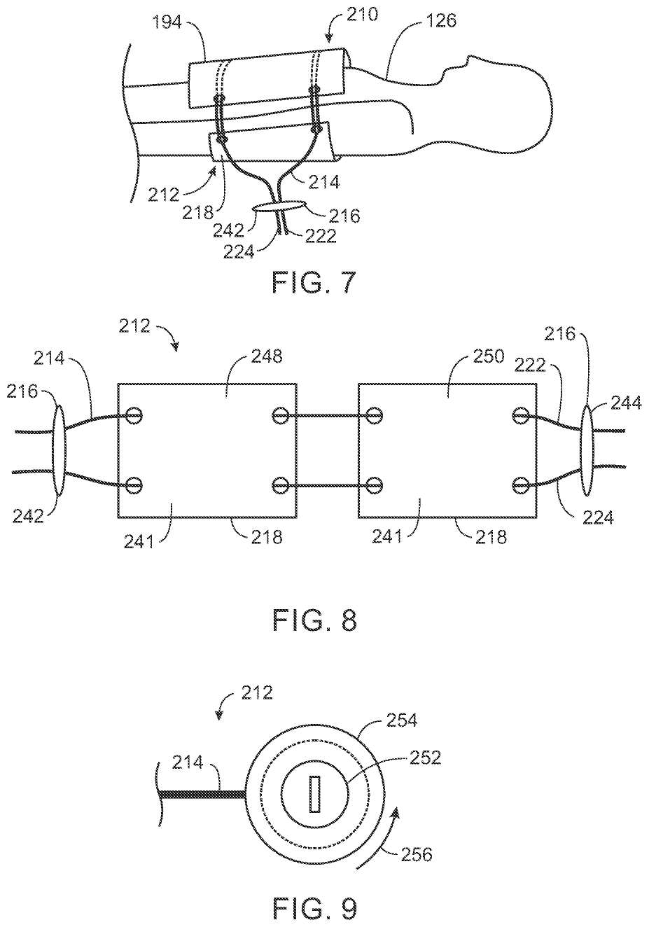

FIG. 7 is a schematic diagram of a portion of the adaptive compression system or integrated positioning system 212 in FIG. 5 disposed about the subject 126 (e.g., patient) to be imaged. The body coil 210 (and the flexible enclosure 194) are configured to be disposed about a first side of the portion of the subject 126 to be imaged. As depicted, the body coil 210 (and the flexible enclosure 194) are disposed on a chest region of the subject 126. The flexible retaining element 218 is configured to be disposed about a second side of the portion of the subject 126 to be imaged. As depicted, the flexible retaining element 218 is disposed about on the back region of the subject. The first cord or band 222 and/or the second cord or band 224 may be adjusted, via the respective tension adjustment mechanisms 242, 244 (not shown), to restrain the body coil 210 closer to the portion of the subject 126 to be imaged by tightening it around the body.

In certain embodiments, the flexible retaining element 24 may include more than one piece. FIG. 8 is a schematic diagram of a portion of the adaptive compression system or integrated positioning system 212 in FIG. 5 (e.g., with a multi-piece flexible retaining element 218). As depicted, the flexible retaining element 218 includes a first piece 248 and a second piece 250. Both the first piece 248 and the second piece 250 are flat flexible pieces that are configured to bend around a curved surface of a portion of a body.

In certain embodiments, a different mechanism may be utilized for the tension adjustment mechanism 216. FIG. 9 is a schematic diagram of a portion of the tension adjustment mechanism 216. As depicted, the tension adjustment mechanism 212 is coupled to the elastic tension element 214. In certain embodiments, the tension adjustment mechanism 216 includes a dial 252 coupled to a spool 254. The dial 252 is configured to be turned (e.g., in circumferential direction 256) to wind the elastic tension element 214 on to the spool 254 to tighten the body coil (and the flexible enclosure) about the portion of the subject to be imaged.

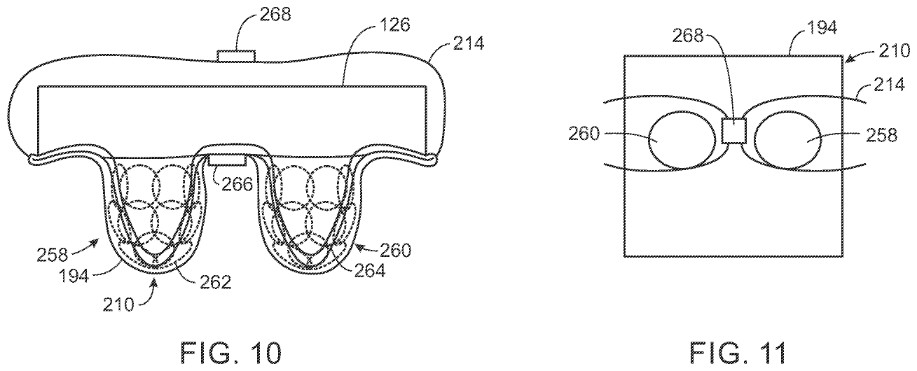

FIG. 10 is a schematic diagram of a top view of the body coil 210 having the adaptive compression system or integrated positioning system 212 in FIG. 4 (e.g., configured for use with breasts) disposed on a chest region (e.g., having breasts) of the subject 126 (e.g., patient). FIG. 11 is a schematic diagram of a rear view of the body coil 210 having the adaptive compression system or integrated positioning system 212 in FIG. 10 that is not disposed on the subject 126. As depicted in FIG. 10, the body coil 210 (and the flexible enclosure 194) are disposed on the chest region (including the breasts) of the subject 126. The body coil 210 (and the flexible enclosure 194) include cup regions 258, 260 configured to be disposed about respective breasts of the subject 126. A portion of the elastic tension element 214 is disposed within the flexible enclosure 194. In particular, the portion of the elastic tension element 214 disposed within the flexible enclosure 194 includes a first net-like region of cordage 262 in the cup region 258 and a second net-like region of cordage 264 in the cup region 260.

The adaptive compression system 212 includes a first tension adjustment mechanism 266 disposed outside the flexible enclosure 194 of the body coil 210. A portion of the elastic tension element 214 coupled to both the first net-like region of cordage 262 and the second net-like region of cordage 264 extends outside the flexible enclosure 194 and is coupled to the first tension adjustment mechanism 266. The first tension adjustment mechanism 266 is configured to enable selective adjustment (e.g., different adjustment) of the first net-like region of cordage 26 and the second net-like region of cordage with respect to tightening or loosening engagement of the cup regions 258, 260 with the respective breasts. Thus, the adaptive compression system 212 is configured to handle different cup sizes on the same individual or between different individuals.

A portion of the elastic tension element 214 external to the flexible enclosure 194 extends around the sides of the subject 126 and is coupled to a second tension adjustment mechanism 268 along the back of the subject 126. The second tension adjustment mechanism 268 is configured to enable general tightening or loosening of the body coil 210 (and the flexible enclosure 194) on the chest region (and the breasts) of the subject 126.

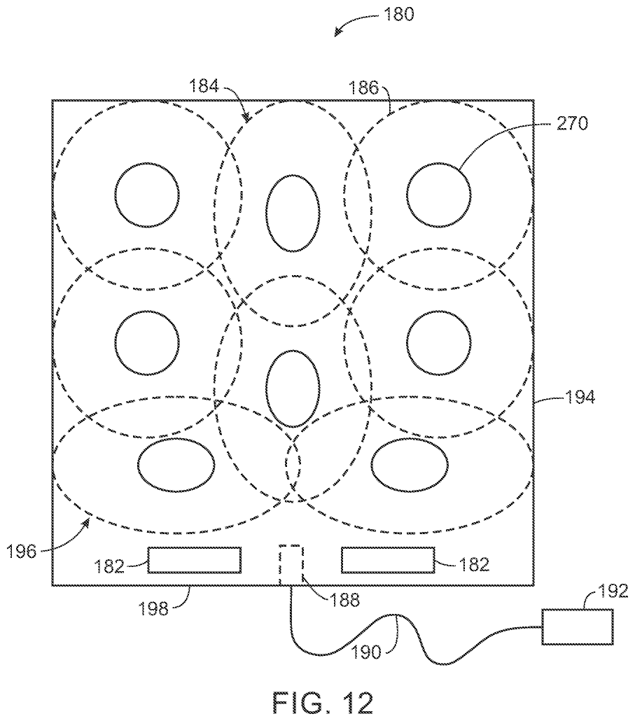

FIG. 12 is a schematic diagram of the radio frequency (RF) coil assembly 180 to be utilized with an adaptive compression system or integrated positioning system (e.g., with the flexible enclosure 194 having openings). The radio frequency coil assembly 180 is as described in FIG. 2. As depicted in FIG. 12, the flexible enclosure 194 includes a plurality of holes or openings 270. The plurality of holes or openings 270 is configured to increase a flexibility of the radio frequency coil assembly 180 (and the flexible enclosure 194). Each hole or opening 270 may be radially located within the loop 186. In certain embodiments, a respective hole or opening 270 may be located within each loop 186. In certain embodiments, a respective hole or opening 270 may only be located within some of the loops 186. In certain embodiments, the number of holes or openings 270 may vary. In certain embodiments, the shapes of the plurality of holes or openings 270 may vary. In certain embodiments, the shapes of the plurality of holes or openings 270 may vary on the same radio frequency coil assembly 180. In certain embodiments, the shapes of the plurality of holes or openings 270 may vary between different radio frequency coil assemblies 180.

Technical effects of the disclosed subject matter include providing an adaptive compression system (e.g. integrated adaptive compression system) or integrated positioning system for retaining a body coil (e.g., a radio frequency coil assembly or radio frequency receiving coil) on a subject to be imaged. The adaptive compression system is adjustable so that the body coil can be pulled as tight or as loosely as needed while the cordage layout allows for customized contours around a wide range of anatomical regions. Technical effects of the disclosed subject matter also include providing a more comfortable positioning of the body coil on the body. The cordage can be loosened or repositioned as needed for imaging and patient comfort. The disclosed adaptive compression system can be oriented in different locations. Technical effects of the disclosed subject matter further includes providing a much faster and streamlined configuration. Thus, the setup time is reduced and the throughput for use of the magnetic resonance scanner can be increased. Also, the need for use of bulker positioners is eliminated. Technical effects of the disclosed subject matter even further includes increasing patient safety due to cinched body coil being put tighter against the body away from the bore of the magnetic resonance scanner. Thus, the body coil is less likely to heat as much (e.g., due to field inhomogeneities at the edge of the bore). This is especially useful for parts of the anatomy such as shoulders and hips that are already in close proximity to the bore. Technical effects of the disclosed subject matter yet further includes increasing image quality due to a higher signal-to-noise ratio since the loops of the body coil will be closer to the body. Technical effects of the disclosed subject matter still further includes providing a positioning system that is easier to maintain and to clean.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A radio frequency receiving coil assembly for a magnetic resonance imaging system, comprising:
a flexible enclosure; and
a radio frequency coil disposed within the flexible enclosure, wherein the radio frequency coil comprises a plurality of flexible loops having a malleable conductor, and the flexible enclosure is configured to be disposed on a first side of a portion of a subject to be imaged; and
an adaptive compression system, comprising:
a flexible retaining element separate from the flexible enclosure that is configured to be disposed on a second side of the portion of the subject to be imaged;
at least one band coupled to both the flexible enclosure and the flexible retaining element; and
a tension adjustment mechanism coupled to the at least one band and configured to adjust a tension of the at least one band to adjust how tight the flexible enclosure is disposed about the portion of the subject to be imaged.

2. The radio frequency receiving coil assembly of claim 1, wherein at least a portion of the at least one band is disposed within the flexible enclosure.

3. The radio frequency receiving coil assembly of claim 1, wherein the tension adjustment mechanism is coupled to the at least one band outside the flexible enclosure.

4. The radio frequency receiving coil assembly of claim 1, wherein the tension adjustment mechanism is configured, via the at least band, to pull the flexible enclosure and the flexible retaining element closer together about the portion of the subject to be imaged.

5. The radio frequency receiving coil assembly of claim 1, wherein the at least one band comprises a first band and a second band coupled to both the flexible enclosure and the flexible retaining element.

6. The radio frequency receiving coil assembly of claim 5, wherein the flexible enclosure comprises a first end and a second end opposite the first end, the first band and the second band are respectively disposed closer to the first end and the second end, and the tension adjustment mechanism is coupled to both the first band and the second band and is configured to adjust the tension of both the first band and the second band.

7. The radio frequency receiving coil assembly of claim 1, wherein the at least one band is configured to be removed and to be replaced.

8. The radio frequency receiving coil assembly of claim 1, wherein the at least one band comprises twine, an elastic cord, or an elastic band.

9. The radio frequency receiving coil assembly of claim 1, wherein the tension adjustment mechanism comprises a dial coupled to a spool, wherein the dial is configured to be turned to wind the at least one band on to the spool to tighten the flexible enclosure about the portion of the subject to be imaged.

10. The radio frequency receiving coil assembly of claim 1, wherein the tension adjustment mechanism comprises a cord lock stopper.

11. The radio frequency receiving coil assembly of claim 1, wherein the adaptive compression system is configured to enable positioning of the flexible enclosure about curved surfaces of different parts of an anatomy of the subject to be imaged.

12. A radio frequency receiving coil assembly for a magnetic resonance imaging system, comprising:
a flexible enclosure; and
a radio frequency coil disposed within the flexible enclosure, wherein the radio frequency coil comprises a plurality of flexible loops having a malleable conductor, and the flexible enclosure is configured to be disposed on a first side of a portion of a subject to be imaged; and
an integral adaptive compression system, comprising:
a flexible retaining element separate from the flexible enclosure that is configured to be disposed on a second side of the portion of the subject to be imaged;
cordage coupled to both the flexible enclosure and the flexible retaining element, wherein at least a portion of the cordage is disposed within the flexible enclosure; and
a tension adjustment mechanism coupled to the cordage and configured to adjust a tension of the cordage to adjust how tight the flexible enclosure is disposed about the portion of the subject to be imaged.

13. The radio frequency receiving coil assembly of claim 12, wherein the portion of the subject to be imaged is a chest having breasts, and wherein the integral adaptive compression system is configured to enable positioning of the flexible enclosure about breasts of different sizes.

14. The radio frequency receiving coil assembly of claim 13, wherein the cordage comprises a first net-like region of cordage within the flexible enclosure and a second net-like region of cordage within the flexible enclosure that are configured to be respectively disposed about a respective breast of the breasts.

15. The radio frequency receiving coil assembly of claim 12, wherein the tension adjustment mechanism is coupled to the cordage outside the flexible enclosure.

16. The radio frequency receiving coil assembly of claim 12, wherein the tension adjustment mechanism is configured, via the cordage, to pull the flexible enclosure and the flexible retaining element closer together about the portion of the subject to be imaged.

17. The radio frequency receiving coil assembly of claim 12, wherein the cordage comprises a first band and a second band coupled to both the flexible enclosure and the flexible retaining element.

18. The radio frequency receiving coil assembly of claim 17, wherein the flexible enclosure comprises a first end and a second end opposite the first end, the first band and the second band are respectively disposed closer to the first end and the second end, and the tension adjustment mechanism is coupled to both the first band and the second band and is configured to adjust the tension of both the first band and the second band.

19. The radio frequency receiving coil assembly of claim 12, wherein the integral adaptive compression system is configured to enable positioning of the flexible enclosure about curved surfaces of different parts of an anatomy of the subject to be imaged.

20. A radio frequency receiving coil assembly for a magnetic resonance imaging system, comprising:
a flexible enclosure; and
a radio frequency coil disposed within the flexible enclosure, wherein the radio frequency coil comprises a plurality of flexible loops having a malleable conductor, and the flexible enclosure is configured to be disposed on a chest having breasts of a subject to be imaged; and
an integral adaptive compression system, wherein the integral adaptive compression system is configured to enable positioning of the flexible enclosure about breasts of different sizes, wherein the integral adaptive compression system comprises:
a flexible retaining element separate from the flexible enclosure that is configured to be disposed on a back of the subject to be imaged;
cordage coupled to both the flexible enclosure and the flexible retaining element, wherein the cordage comprises a first net-like region of cordage within the flexible enclosure and a second net-like region of cordage within the flexible enclosure that are configured to be respectively disposed about a respective breast of the breasts; and
a tension adjustment mechanism coupled to the cordage and configured to adjust a tension of the cordage to adjust how tight the flexible enclosure is disposed about the chest of the subject to be imaged.
